(19) United States
(12) Patent Application Publication
(10) Pub. No.: US 2026/0191611 A1
(43) Pub. Date: Jul. 9, 2026

(54) MULTI CATHETER METHOD OF PERFORMING A ROBOTIC NEUROVASCULAR PROCEDURE

(71) Applicant: Imperative Care, Inc., Campbell, CA (US)

(72) Inventors: Lilip Lau, Los Altos, CA (US); Robert Hitchcock, Los Gatos, CA (US); Kyle Bartholomew, Campbell, CA (US); David Gonzalez, Campbell, CA (US)

(21) Appl. No.: 19/547,287

(22) Filed: Feb. 23, 2026

Related U.S. Application Data
(63) Continuation of application No. 17/527,456, filed on Nov. 16, 2021, now Pat. No. 12,558,175.
(60) Provisional application No. 63/232,444, filed on Aug. 12, 2021.

(51) Int. Cl. A61B 34/37; A61B 5/00; A61B 17/22; A61B 34/00; A61B 34/30; A61M 25/00; A61M 25/01; A61M 25/09

(52) U.S. Cl. CPC A61B 34/37; A61B 5/6852; A61B 17/22; A61B 34/30; A61B 34/71; A61M 25/0097; A61M 25/0127; A61M 25/09; A61B 2017/22038; A61B 2017/22079; A61B 2034/301; A61B 2034/715; A61B 2562/0261; A61M 25/002; A61M 25/0113; A61M 2205/3306; A61M 2205/332; A61M 2205/3331; A61M 2205/3592; A61M 2205/583

(57) ABSTRACT

A method of performing a neurovascular procedure includes the steps of providing an access assembly comprising a guidewire, access catheter and guide catheter; coupling the access assembly to a robotic drive system; and driving the access assembly to achieve supra-aortic vessel access. The guide wire and the access catheter are decoupled from the access assembly. A procedure assembly includes at least a guidewire and a procedure catheter. The procedure assembly is coupled to the robotic drive system; and used to perform a neurovascular procedure.

### CROSS-REFERENCE TO RELATED APPLICATIONS

Any and all applications for which a foreign or domestic priority is identified in the Application Data Sheet as filed with the present application are hereby incorporated by reference under 37 CFR 1.57. The present application is a continuation of U.S. patent application Ser. No. 17/527456, filed Nov. 16, 2021, titled “MULTI CATHETER METHOD OF PERFORMING A ROBOTIC NEUROVASCULAR PROCEDURE,” which claims the benefit of U.S. Provisional Application No. 63/232,444, filed Aug. 12, 2021, the entirety of which are hereby incorporated by reference herein.

### BACKGROUND OF THE INVENTION

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neuro interventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to frictional interplay between coaxial shafts and the patient's vasculature. Supra-aortic access necessary to reach the neurovascular is challenging to achieve, especially Type III arches.

Thus, there remains a need for a supra-aortic access system that addresses some or all of these challenges, and increases the availability of neurovascular procedures. Preferably, the system is additionally capable of driving devices further distally through the supra-aortic access to accomplish procedures in the intracranial vessels.

### SUMMARY OF THE INVENTION

There is provided in accordance with one aspect of the present invention a supra-aortic access robotic control system. The system comprises a guidewire hub configured to adjust each of an axial position and a rotational position of a guidewire; a guide catheter hub configured to adjust a guide catheter in an axial direction; and an access catheter hub configured to adjust each of an axial position and a rotational position of an access catheter, and also to laterally deflect a distal deflection zone of the access catheter. The guidewire hub may additionally be configured to laterally deflect a distal portion of the guidewire.

There may also be provided a procedure catheter hub configured to manipulate a procedure catheter. Following robotic placement of the guidewire, access catheter and guide catheter such that the guide catheter achieves supra-aortic access, the guidewire and access catheter may be proximally withdrawn, and the procedure catheter advanced through and beyond the guide catheter to reach a neurovascular treatment site. The procedure catheter may be an aspiration catheter; an embolic deployment catheter; a stent deployment catheter; a flow diverter deployment catheter, an access catheter; a diagnostic angiographic catheter; a guiding catheter, an imaging catheter, a physiological sensing/measuring catheter, an infusion or injection catheter, a balloon catheter or a stent retriever.

The control system may further comprise a driven magnet on each of a guidewire hub, an access catheter hub, and a guide catheter hub, configured to cooperate with corresponding drive magnets such that the driven magnet moves in response to movement of the corresponding drive magnet. The drive magnets may each be independently axially movably carried by a support table. The drive magnets may be located outside of the sterile field, separated from the driven magnets by a barrier, and the driven magnets may within the sterile field. The barrier may comprise a tray made from a thin polymer membrane, or any membrane of non-ferromagnetic material.

The control system may further comprise a control console which may be connected to the support table or may be located remotely from the support table. The position of each driven magnet and corresponding hub is movable in response to manual manipulation of a guidewire drive control, access catheter drive control or procedure catheter drive control on the console.

The control system may further comprise a processor for controlling the position of the drive magnets. The processor may be in wired communication with the control console, or in wireless communication with the control console. The driven magnets may be configured to remain engaged with the corresponding drive magnets until application of a disruption force of at least about 300 grams.

There is also provided a robotically driven interventional device. The device comprises an elongate, flexible body, having a proximal end and a distal end. A hub is provided on the proximal end. At least one rotatable roller is provided on a first surface of the hub; and at least one magnet is provided on the first surface of the hub. The roller may extend further away from the first surface than the magnet. The hub may be further provided with at least a second roller.

Any of the guidewire hub, access catheter hub and procedure catheter hub may be further provided with a rotational drive, for rotating the corresponding interventional device with respect to the hub. The hub may be further provided with an axial drive mechanism to distally advance or proximally retract a control element extending axially through the interventional device, to adjust a characteristic such as shape or flexibility of the interventional device. The control element may be an axially movable tubular body or wire such as a pull wire extending through the interventional device to, for example, a distal deflection zone.

There is also provided a control system for controlling movement of interventional devices. In one configuration, the control system comprises a guidewire control, configured to control axial travel and rotation of a guidewire; an access catheter control, configured to control axial and rotational movement of an access catheter; and a guide catheter control, configured to control axial movement of a guide catheter.

The control system may further comprise a deflection control, configured to control deflection of the access catheter, and may be configured for wired or wireless communication with a robotic catheter drive system.

The control system may be configured to independently control the three or more hubs in a variety of modes. For example, two or more hubs may be selectively ganged together so that they drive the respective devices simultaneously and with the same motion. Alternatively, the control system may be configured to drive respective devices simultaneously but with different motions.

The control system may further comprise a physician interface for operating the control system. The physician interface may be carried by a support table having a robotic interventional device drive system. Alternatively, the physician interface for operating the control system may be carried on a portable, handheld device or desktop computer, and may be located in the same room as the patient, the same facility as the patient, or in a remote facility.

The control system may further comprise a graphical user interface with at least one display for indicating the status of at least one device parameter, and/or indicating the status of at least one patient parameter.

There is also provided a sterile packaging assembly for transporting interventional devices to a robotic surgery site. The packaging assembly may comprise a base and a sterile barrier configured to enclose a sterile volume. At least one interventional device may be provided within the sterile volume, the device including a hub and an elongate flexible body. The hub may include at least one magnet and at least one roller configured to roll on the base.

In one implementation, the sterile barrier is removably attached to the base to define the enclosed volume between the sterile barrier and the base. In another implementation, the sterile barrier is in the form of a tubular enclosure for enclosing the sterile volume. The tubular enclosure may surround the base and the at least one interventional device, which are within the sterile volume.

The hub may be oriented within the packaging such that the roller and the magnet face the base. Alternatively, the base may be in the form of a tray having an elongate central axis. An upper, sterile field side of the tray may have an elongate support surface for supporting and permitting sliding movement of one or more hubs. At least one and optionally two elongate trays may be provided, extending parallel to the central axis. At least one hub and interventional device may be provided in the tray, and the sterile tray with sterile hub and interventional device may be positioned in a sterile volume defined by a sterile barrier.

The base may be configured to reside on a support table adjacent a patient, with an upper surface of the base within a sterile field and a lower surface of the base outside of the sterile field.

Any of the hubs disclosed herein may further comprises a fluid injection port and/or a wireless RF transceiver. The hub may comprise a visual indicator, for indicating the presence of a clot. The visual indicator may comprise a clot chamber having a transparent window. A filter may be provided in the clot chamber.

Any of the hubs disclosed herein may further comprise a sensor for detecting a parameter of interest such as the presence of a clot. The sensor, in some instances, may be positioned on a flexible body. The sensor may comprise a pressure sensor or an optical sensor. In some embodiments, the sensor may comprise one or more of a force sensor, a temperature sensor, and/or an oxygen sensor. In some embodiments, the sensor may comprise a Fiber Bragg grating sensor. For example, a Fiber Bragg grating sensor (e.g., an optical fiber) may detect strain locally that can facilitate the detection and/or determination of force being applied. The device may further include a plurality of sensors. The plurality of sensors may each comprise one or more of any type of sensor disclosed herein. In some embodiments, a plurality (e.g., 3 or more) of sensors (e.g., Fiber Bragg grating sensors) may be distributed around a perimeter to facilitate the detection and/or determination of shape. The position of the device, in some instance, may be determined through the use of one or more sensors to detect and/or determine the position. For example, one or more optical encoders may be located in or proximate to one or more the motors that drive linear motion such that the optical encoders may determine a position.

There is also provided a method of performing a neurovascular procedure, in which a first phase includes robotically achieving supra-aortic access, and a second phase includes manually or robotically performing a neurovascular procedure via the supra-aortic access. The method comprises the steps of providing an access catheter having an access catheter hub; coupling the access catheter hub to a hub adapter movably carried by a support table; driving the access catheter in response to movement of the hub adapter along the table until the access catheter is positioned to achieve supra-aortic access. The access catheter and access catheter hub may then be decoupled from the hub adapter; and a procedure catheter hub having a procedure catheter may then be coupled to the hub adapter.

The method may additionally comprise advancing the procedure catheter hub to position a distal end of the procedure catheter at a neurovascular treatment site. The driving the access catheter step may comprise driving the access catheter distally through a guide catheter. The driving the access catheter step may include the step of laterally deflecting a distal region of the access catheter to achieve supra-aortic access.

There is also provided a method of performing a neurovascular procedure, comprising the steps of providing an access assembly comprising a guidewire, access catheter and guide catheter. The access assembly may be releasably coupled to a robotic drive system. The access assembly may be driven by the robotic drive system to achieve access to a desired point, such as to achieve supra-aortic access. The guide wire and the access catheter may then be decoupled from the access assembly, leaving the guide catheter in place. A procedure assembly may be provided, comprising at least a guidewire and a first procedure catheter. The procedure assembly may be releasably coupled to the robotic drive system; and a neurovascular procedure may be accomplished using the procedure assembly. A second procedure catheter may also be provided, for extending through the first procedure catheter to a treatment site.

The coupling the access assembly step may comprise magnetically coupling a hub on each of the guidewire, access catheter and guide catheter, to separate corresponding couplers carrying corresponding drive magnets independently movably carried by the drive table. The procedure assembly may comprise a guidewire, a first catheter and a second catheter. The guidewire and first catheter may be positioned concentrically within the second catheter. The procedure assembly may be advanced as a unit through at least a portion of the length of the guide catheter, and the procedure may comprise a neurovascular thrombectomy.

Additional features and advantages of the present invention are disclosed in U.S. Provisional Application No. 63/232,444, the entirety of each of which is hereby incorporated by reference herein.

### BRIEF DESCRIPTION OF THE DRAWINGS

FIG. 1 is a schematic perspective view of an interventional setup having an imaging system, a patient support table, and a robotic drive system in accordance with the present invention.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F Show an alternate sterile barrier in the form of a shipping tray having one or more storage channels for carrying interventional devices.

FIG. 4 is a schematic elevational cross section through a hub adapter having a drive magnet separated from an interventional device hub and driven magnet by a sterile barrier.

FIGS. 5A and 5B schematically illustrate a three interventional device and a four interventional device assembly.

FIG. 6 is a perspective view of a support table.

FIG. 7 is a close-up view of the motor drive end of a support table.

FIG. 8 is an elevational cross section through a motor and belt drive assembly.

FIG. 9 is a close-up view of a pulley end of the support table.

FIG. 10 is an elevational cross section through a belt pully.

FIG. 11 is a side elevational cross-section through a distal portion of a catheter such as any of those shown in FIGS. 5A and 5B.

FIGS. 12A and 12B schematically illustrate a force sensor integrated into the sidewall of the catheter.

FIGS. 13A and 13B schematically illustrate a sensor for measuring elastic forces at the magnetic coupling between the hub and corresponding carriage.

FIG. 14 schematically illustrates a dual encoder torque sensor for use with a catheter of the present invention.

FIG. 15 illustrates a clot capture and visualization device that can be integrated into a hub and/or connected to an aspiration line.

### DETAILED DESCRIPTION OF THE PREFERRED EMBODIMENT

The present invention provides a system for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations of the invention, the system may additionally be configured to robotically gain intra-cranial vascular access and to perform an aspiration thrombectomy or other neuro vascular procedure.

A drive table is positioned over or alongside the patient, and configured to axially advance, retract, and in some cases rotate and/or laterally deflect two or three or more different (e.g., concentrically or side by side oriented) intravascular devices. Each device has a proximal end attached to a unique hub, sometimes referred to as a “puck”. The hub is moveable along a path along the surface of the drive table to advance or retract the interventional device as desired. Each hub may also contain mechanisms to rotate or deflect the device as desired, and is connected to fluid delivery tubes (not shown) of the type conventionally attached to a catheter hub. Each hub is in electrical communication with an electronic control system, either via hard wired connection, RF wireless connection or a combination of both.

Each hub is independently movable across the surface of a sterile field barrier membrane carried by the drive table. Each hub is releasably magnetically coupled to a unique drive carriage on the table side of the sterile field barrier. The drive system independently moves each hub in a proximal or distal direction across the surface of the barrier, to move the corresponding interventional device approximately or distally within the patient's vasculature.

The carriages on the drive table which magnetically couple with the hubs to provides linear motion actuation are universal. Functionality of the catheters/guidewire are provided based on what is contained in the hubs and the shaft designs. This allows flexibility to configure the system to do a wide range of procedures using a wide variety of interventional devices on the same drive table.

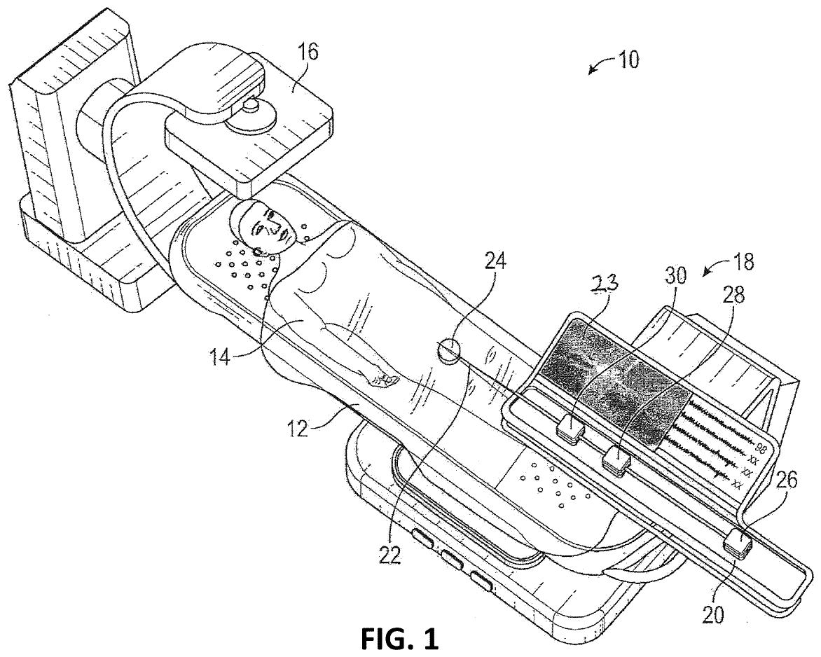

FIG. 1 is a schematic perspective view of an interventional setup 10 having a patient support table 12 for supporting a patient 14. An imaging system 16 may be provided, along with a robotic interventional device drive system 18 in accordance with the present invention.

The drive system 18 may include a support table 20 for supporting, for example, a guidewire hub 26, an access catheter hub 28 and a guide catheter hub 30. In the present context, the term ‘access’catheter can be any catheter having a lumen with at least one distally facing or laterally facing distal opening, that may be utilized to aspirate thrombus, provide access for an additional device to be advanced therethrough or there along, or to inject saline or contrast media or therapeutic agents.

More or fewer interventional device hubs may be provided depending upon the desired clinical procedure. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, pulmonary airways, treatment sites reached via trans ureteral or urethral or fallopian tube navigation, or other hollow organs or structures in the body.

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control, and may also be configured to laterally deflect a deflectable portion of the access catheter, in response to manipulation of a deflection control.

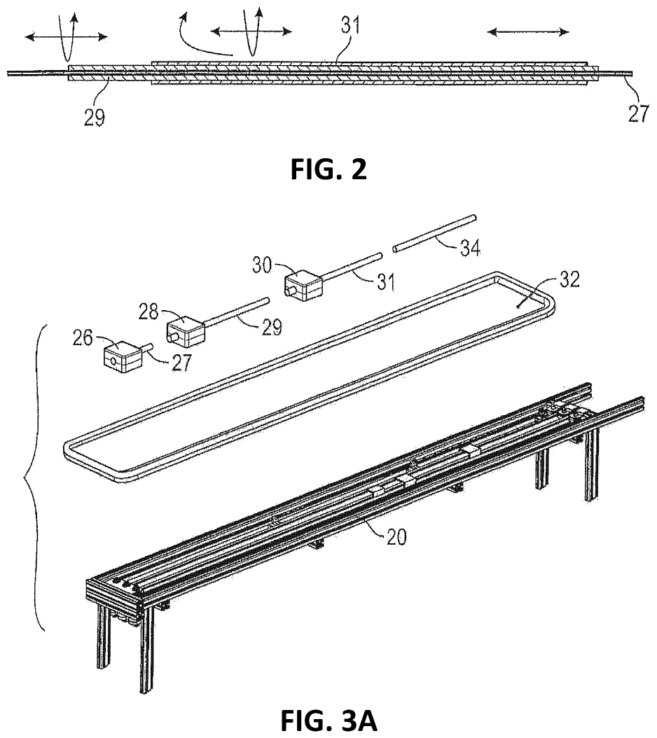

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

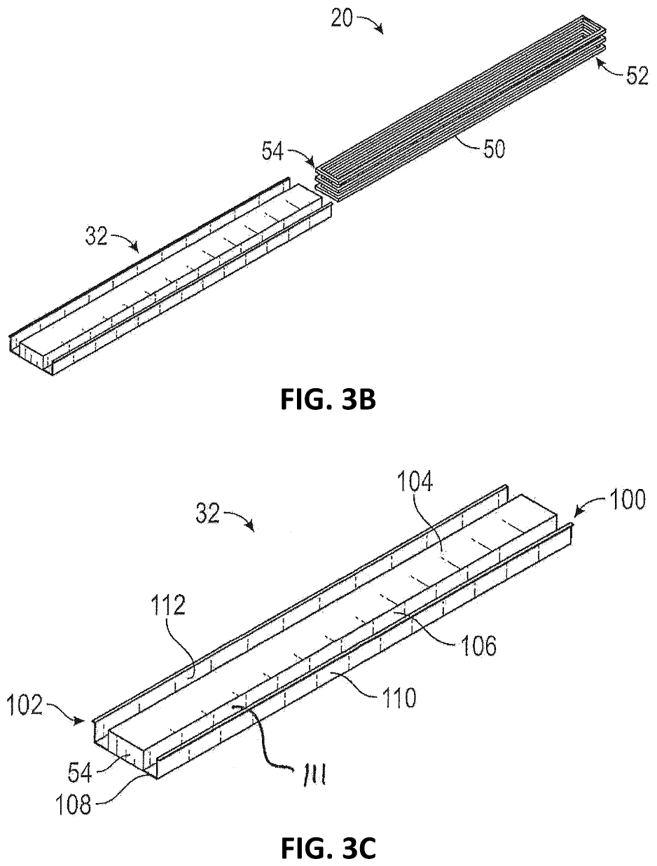

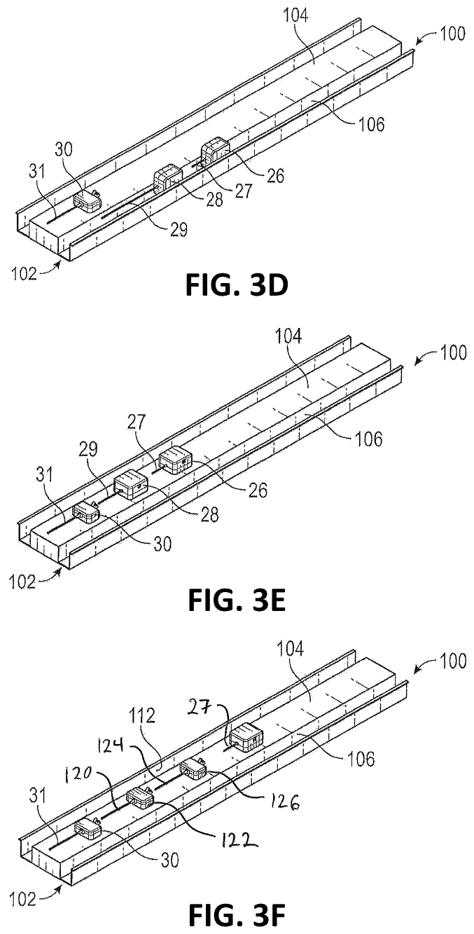

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti buckling zone for resisting buckling of the portion of the interventional devices spanning the distance between the support table 20 and the femoral artery access point 24. The anti-buckling feature 34 may comprise a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 cm or 10 cm but typically no more than about 130 cm or about 100 cm or about 50 cm or about 30 cm to support the device between the hub and the access point 24 on the patient. Reinforcement may be accomplished by embedding at least one or two or more axially extending elements into the wall, such as elongate wires or ribbons. Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, the wall thickness or diameter of the interventional device can be increased in the anti-buckling zone.

The interventional device hubs may be separated from the support table 20 by sterile barrier 32. Sterile barrier 32 may comprise a thin plastic membrane such as PET. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices.

Referring to FIGS. 3B and 3C, there is illustrated a sterile barrier 32 in the form of a pre-shaped tray, for fitting over an elongate support table 20. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration. The length of support surface 104 will typically be at least about 150 cm or about 180 cm in a linear drive table. Shorter lengths may be utilized in a system configured to advance the drive couplers along an arcuate path.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs. First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guide wire hub 26 and guide wire 27 are illustrated residing within the first channel 106 such as before introduction through the guide catheter 31 or following removal from the guide catheter 31. The length of the catheters has been cut down to simplify the drawing.

The interventional devices may be positioned within the channel 106 and enclosed in a sterile barrier for shipping. The sterile barrier containing the sterile interventional devices may be contained within a second, outer sealed container such as a membrane pouch, which may be a second, outer sterile barrier At the clinical site, an upper panel of the sterile barrier may be removed, or an outer tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E the interventional devices configured in an access assembly for aortic access, following coupling of the access assembly to the corresponding carriages beneath the sterile barrier. The access assembly may be pre-assembled with the guidewire fully advanced through the access catheter which is in turn fully advanced through the guide catheter. This access assembly may be lifted out of the channel 106 as a unit and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guide wire hub 26 is positioned most proximally, in order to allow the guide wire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guide wire 27 extends through at least a portion of the second procedure catheter 124 in a rapid exchange version of second procedure catheter 124, or the entire length of second procedure catheter 124 in an over the wire implementation.

In one commercial execution, a preassembled access assembly (guide catheter, access catheter and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure.

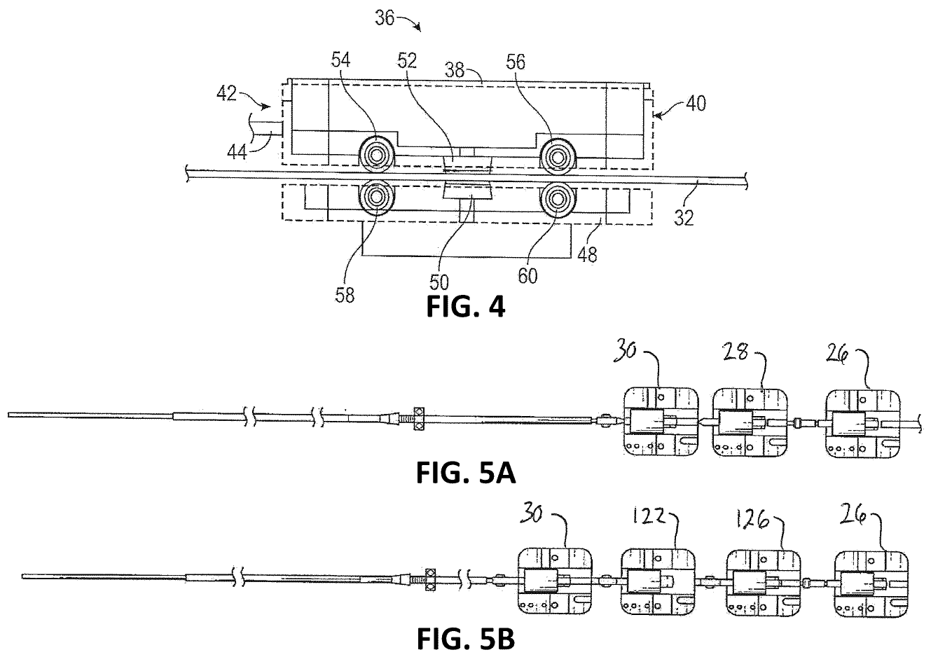

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions. The hub adapter 48 includes at least one drive magnet 50 configured to couple with a driven magnet 52 carried by the hub 36. This provides a magnetic coupling between the drive magnet 50 and driven magnet 52 through the sterile barrier 32 such that the hub 36 and associated interventional device is moved across the top of the sterile barrier 32 within the sterile field in response to movement of the hub adapter 48 outside of the sterile field. Movement of the hub adapter is driven by a drive system carried by the support table and described in additional detail below.

To reduce friction in the system, the hub 36 may be provided with at least a first roller 54 and a second roller 56 which may be in the form of wheels or rotatable balls or drums. The rollers space the sterile barrier 32 apart from the surface of the driven magnet 52 by at least about 0.008″ and generally no more than about 0.03″. In some implementations of the invention the space is within the range of from about 0.010″ and about 0.016″. The space between the drive magnet 50 and driven magnet 52 is generally no more than about 0.15″ and in some implementations is no more than about 0.10″ such as within the range of from about 0.085″ to about 0.090″. The hub adapter 48 may similarly be provided with at least a first hub adapter roller 58 and the second hub adapter roller 60, which may be positioned opposite the respective first roller 54 and second roller 56 as illustrated in FIG. 4.

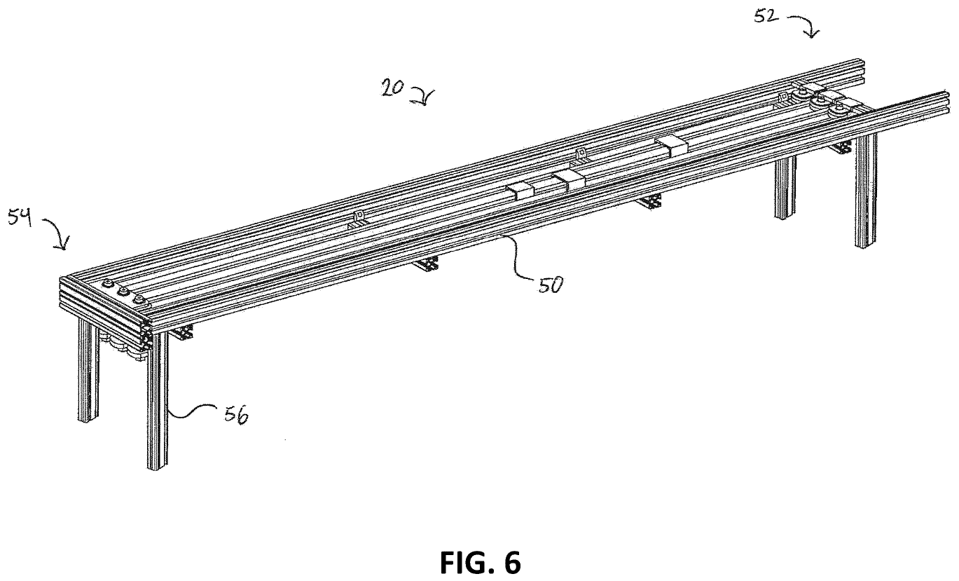

Referring to FIG. 6, there is schematically illustrated one example of a low-profile linear drive support table 20. Support table 20 comprises an elongated frame 50 extending between a proximal end 52 and a distal end 54. At least one support table support 56 is provided to stabilize the support table 20 with respect to the patient (not illustrated). Support 56 may comprise one or more legs or preferably an articulating arm configured to allow movement and positioning of the frame 50 over or adjacent to the patient.

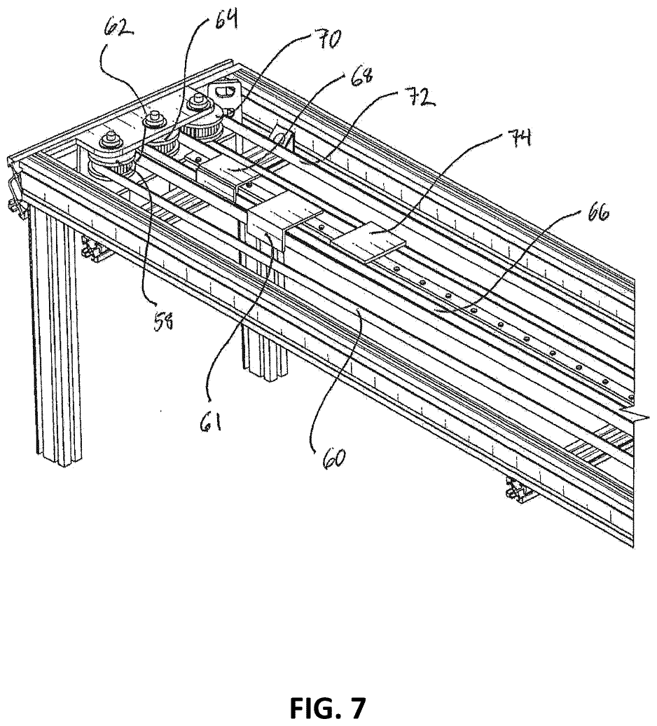

One example of a linear drive table 20 illustrated in FIG. 7 includes three distinct drives. However, two drives or four or more drives may be included depending upon the desired clinical performance. A first drive pulley 58 engages a first drive belt 60. A first carriage bracket 61 is secured to the first drive belt 60 such that rotation of the first drive pulley 58 causes rotation of the first drive belt 60 through an elongate closed loop path. The first carriage bracket 61 may be advanced in a proximal or distal direction along the longitudinal axis of the support table 20 depending upon the direction of rotation of the drive pully 58. In the illustrated implementation, the drive pulley 58 is provided with surface structures such as a plurality of drive pulley teeth 62 for engaging complementary teeth on the first drive belt 60.

A second drive pulley 64 may engage a second drive belt 66 configured to axially move a second carriage bracket 68 along an axial path on the support table 20. A third drive pulley 70 may be configured to drive a third drive belt 72, to advance a third carriage bracket 74 axially along the support table 20. Each of the carriage brackets may be provided with a drive magnet assembly discussed previously but not illustrated in FIG. 7, to form couplers for magnetically coupling to a corresponding driven magnet within the hub of an interventional device as has been discussed.

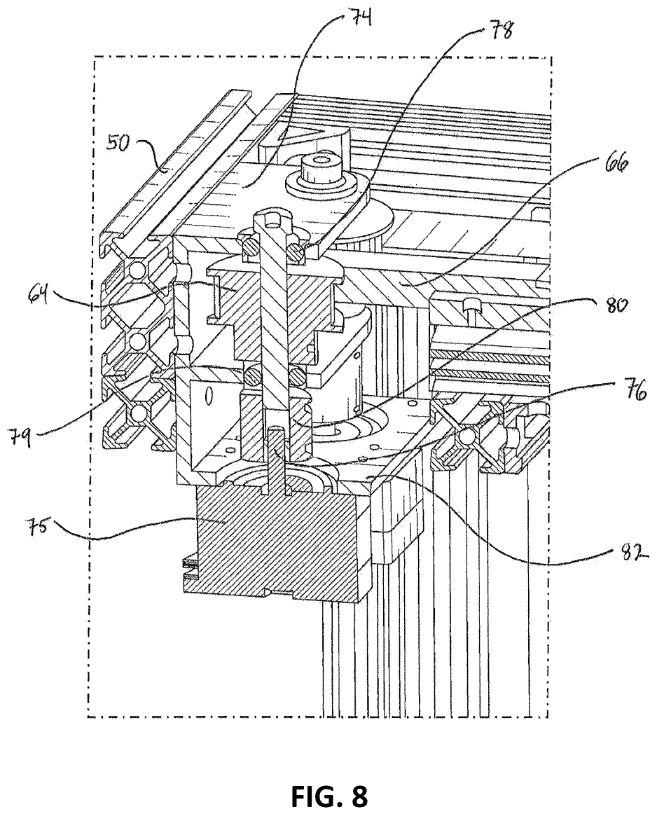

A detail view of a drive system is shown schematically in FIG. 8. A drive support 74 may be carried by the frame 50 for supporting the drive assembly. The second drive pulley 64 is shown in elevational cross section as rotationally driven by a motor 75 via a rotatable shaft 76. The rotatable shaft 76 may be rotatably carried by the support 74 via a first bearing 78, a shaft coupling 80 and second bearing 79. Motor 75 may be stabilized by a motor bracket 82 connected to the drive support 74 and or the frame 50. The belt drive assemblies for the first drive belt 60 and third drive belt 72 maybe similarly constructed and are not further detailed herein.

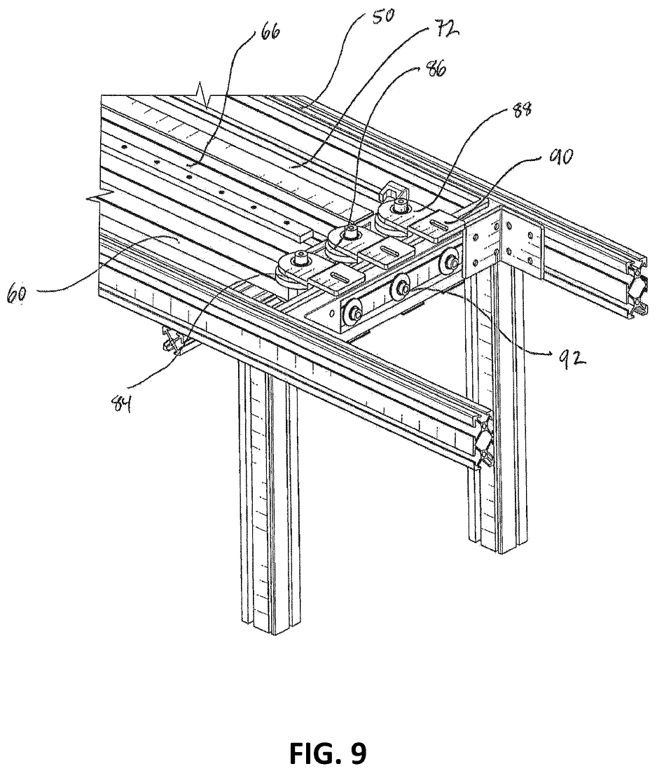

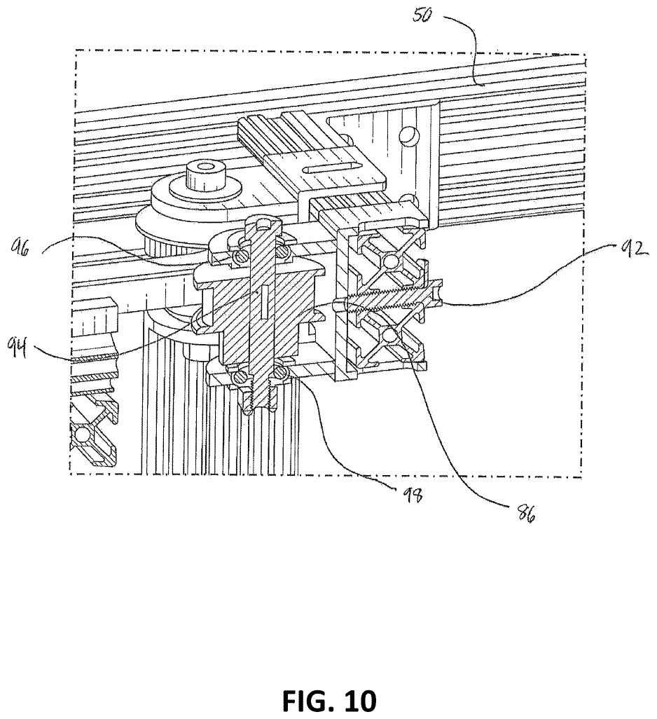

Referring to FIGS. 9 and 10, each of the first second and third drive belts extends around a corresponding first idler pulley 84 second idler pulley 86 and third idler pulley 88. Each idler pulley may be provided with a corresponding tensioning bracket 90, configured to adjust the idler pulleys in a proximal or distal direction in order to adjust the tension of the respective belt. Each tensioning bracket 90 is therefore provided with a tensioning adjustment 92 such as a rotatable screw.

As seen in FIG. 10, the second idler pulley 86, for example, may be carried by a rotatable shaft 94, rotatably secured with respect to the mounting bracket by a first bearing 96 and second bearing 98.

Any of the catheters illustrated, for example, in FIG. 5A, 5B or 11 generally comprise an elongate tubular body extending between a proximal end and a distal functional end. The length and diameter of the tubular body depends upon the desired application. For example, lengths in the area of from about 120 cm to about 140 cm or more are typical for use in femoral access percutaneous transluminal coronary applications. Intracranial or other applications may call for a different catheter shaft length depending upon the vascular access site.

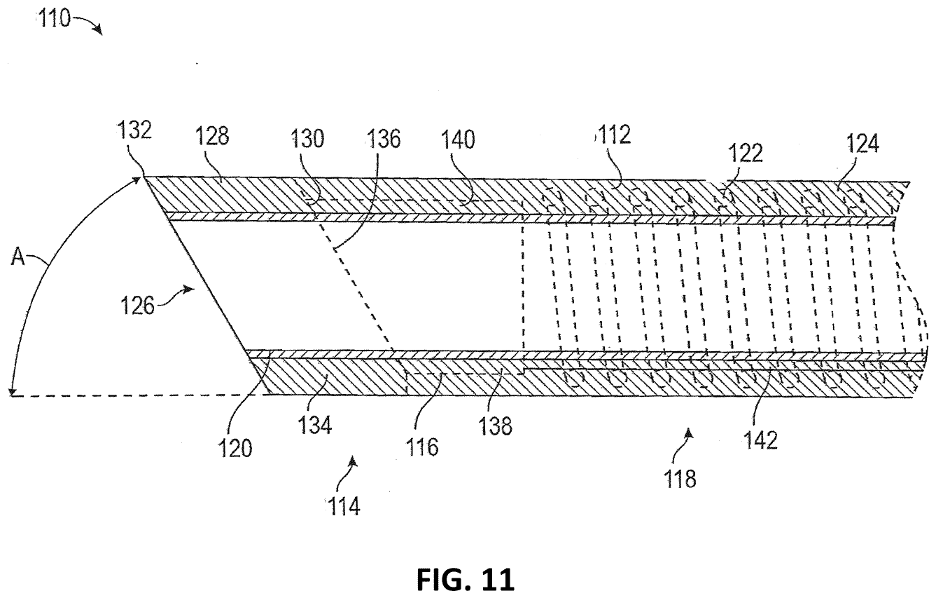

Any of the catheters disclosed herein may be provided with an inclined distal tip. Referring to FIG. 11, distal catheter tip 110 comprises a tubular body 112 which includes an advance segment 114, a marker band 116 and a proximal segment 118. An inner tubular liner 120 may extend throughout the length of the distal catheter tip 110, and may comprise dip coated PTFE.

A reinforcing element 122 such as a braid or spring coil is embedded in an outer jacket 124 which may extend the entire length of the catheter.

The advance segment 114 terminates distally in an angled face 126, to provide a leading side wall portion 128 having a length measured between the distal end 130 of the marker band 116 and a distal tip 132. A trailing side wall portion 134 of the advance segment 114, has an axial length in the illustrated embodiment of approximately equal to the axial length of the leading side wall portion 128 as measured at approximately 180 degrees around the catheter from the leading side wall portion 128. The leading side wall portion 128 may have an axial length within the range of from about 0.1 mm to about 5 mm and generally within the range of from about 1 to 3 mm. The trailing side wall portion 134 may be equal to or at least about 0.1 or 0.5 or 1 mm or 2 mm or more shorter than the axial length of the leading side wall portion 128, depending upon the desired performance.

The angled face 126 inclines at an angle A within the range of from about 45 degrees to about 80 degrees from the longitudinal axis of the catheter. For certain implementations, the angle is within the range of from about 55 degrees to about 65 degrees from the longitudinal axis of the catheter. In one implementation the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105%, and no more than about 130%, in some implementations within the range of from about 110% and about 125% and in one example is about 115% of the area of the corresponding circular port (angle A is 90 degrees).

In the illustrated embodiment, the axial length of the advance segment is substantially constant around the circumference of the catheter, so that the angled face 126 is approximately parallel to the distal surface 136 of the marker band 116. The marker band 116 has a proximal surface approximately transverse to the longitudinal axis of the catheter, producing a marker band 116 having a right trapezoid configuration in side elevational view. A short sidewall 138 is rotationally aligned with the trailing side wall portion 134, and has an axial length within the range of from about 0.2 mm to about 4 mm, and typically from about 0.5 mm to about 2 mm. An opposing long sidewall 140 is rotationally aligned with the leading side wall portion 128. Long sidewall 140 of the marker band 116 is generally at least about 10% or 20% longer than short sidewall 138 and may be at least about 50% or 70% or 90% or more longer than short sidewall 138, depending upon desired performance. Generally, the long sidewall 140 will have a length of at least about 0.5 mm or 1 mm and less than about 5 mm or 4 mm.

The marker band may be a continuous annular structure, or may have at least one and optionally two or three or more axially extending slits throughout its length. The slit may be located on the short sidewall 138 or the long sidewall 140 or in between, depending upon desired bending characteristics. The marker band may comprise any of a variety of radiopaque materials, such as a platinum/iridium alloy, with a wall thickness preferably no more than about 0.003 inches and in one implementation is about 0.001 inches.

The marker band zone of the assembled catheter may have a relatively high bending stiffness and high crush strength, such as at least about 50% or at least about 100% less than proximal segment 18 but generally no more than about 200% less than proximal segment 118. The high crush strength may provide radial support to the adjacent advance segment 114 and particularly to the leading side wall portion 128, to facilitate the functioning of distal tip 132 as an atraumatic bumper during transluminal advance and to resist collapse under vacuum. The proximal segment 118 preferably has a lower bending stiffness than the marker band zone, and the advance segment 114 preferably has even a lower bending stiffness and crush strength than the proximal segment 118.

The advance segment 114 may comprise a distal extension of the outer tubular jacket 124 and optionally the inner liner 120, without other internal supporting structures distally of the marker band 116. Outer jacket 124 may comprise extruded Tecothane. The advance segment 114 may have a bending stiffness and radial crush stiffness that is no more than about 50%, and in some implementations no more than about 25% or 15% or 5% or less than the corresponding value for the proximal segment 118.

The catheter may further comprise an axial tension element or support such as a ribbon or one or more filaments or fibers for increasing the tension resistance and/or influencing the bending characteristics in the distal zone. The tension support may comprise one or more axially extending mono strand or multi strand filaments 142. The one or more tension element 142 may be axially placed inside the catheter wall near the distal end of the catheter. The one or more tension element 142 may serve as a tension support and resist tip detachment or elongation of the catheter wall under tension (e.g., when the catheter is being proximally retracted through a kinked outer catheter or tortuous or narrowed vasculature).

At least one of the one or more tension element 142 may proximally extend along the length of the catheter wall from within about 1.0 cm from the distal end of the catheter to less than about 10 cm from the distal end of the catheter, less than about 20 cm from the distal end of the catheter, less than about 30 cm from the distal end of the catheter, less than about 40 cm from the distal end of the catheter, or less than about 50 cm from the distal end of the catheter.

The one or more tension element 142 may have a length greater than or equal to about 40 cm, greater than or equal to about 30 cm, greater than or equal to about 20 cm, greater than or equal to about 10 cm, or greater than or equal to about 5 cm.

At least one of the one or more tension element 142 may extend at least about the most distal 50 cm of the length of the catheter, at least about the most distal 40 cm of the length of the catheter, at least about the most distal 30 cm or 20 cm or 10 cm of the length of the catheter.

In some implementations, the tension element extends proximally from the distal end of the catheter along the length of the coil 24 and ends proximally within about 5 cm or 2 cm or less either side of a transition between a distal coil and a proximal braid. The tension element may end at the transition without overlapping with the braid.

The one or more tension element 142 may be placed near or radially outside the inner liner 120. The one or more tension element 142 may be placed near or radially inside the braid and/or the coil. The one or more tension element 142 may be carried between the inner liner 120 and the helical coil, and may be secured to the inner liner or other underlying surface by an adhesive prior to addition of the next outer adjacent layer such as the coil. Preferably, the tension element 142 is secured to the marker band 116 such as by adhesives or by mechanical interference. In one implementation, the tension element 142 extends distally beyond the marker band on a first (e.g., inside) surface of the marker band, then wraps around the distal end of the marker band and extends along a second (e.g., outside) surface in either or both a proximal inclined or circumferential direction to wrap completely around the marker band.

When more than one tension element 142 or filament bundles are spaced circumferentially apart in the catheter wall, the tension elements 142 may be placed in a radially symmetrical manner. For example, the angle between two tension elements 142 with respect to the radial center of the catheter may be about 180 degrees. Alternatively, depending on desired clinical performances (e.g., flexibility, trackability), the tension elements 142 may be placed in a radially asymmetrical manner. The angle between any two tension elements 142 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 142 may comprise materials such as Vectran, Kevlar, Polyester, Meta-Para-Aramide, or any combinations thereof. At least one of the one or more tension element 142 may comprise a single fiber or a multi-fiber bundle, and the fiber or bundle may have a round or rectangular (e.g. ribbon) cross section. The terms fiber or filament do not convey composition, and they may comprise any of a variety of high tensile strength polymers, metals or alloys depending upon design considerations such as the desired tensile failure limit and wall thickness. The cross-sectional dimension of the one or more tension element 142, as measured in the radial direction, may be no more than about 2%, 5%, 8%, 15%, or 20% of that of the catheter 10.

The cross-sectional dimension of the one or more tension element 142, as measured in the radial direction, may be no more than about 0.001 inches, no more than about 0.002 inches, no more than about 0.004 inches, no more than about 0.006 inches, no more than about 0.008 inches, or about 0.015 inches.

The one or more tension element 142 may increase the tensile strength of the distal zone of the catheter before failure under tension (e.g. marker band detachment) to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

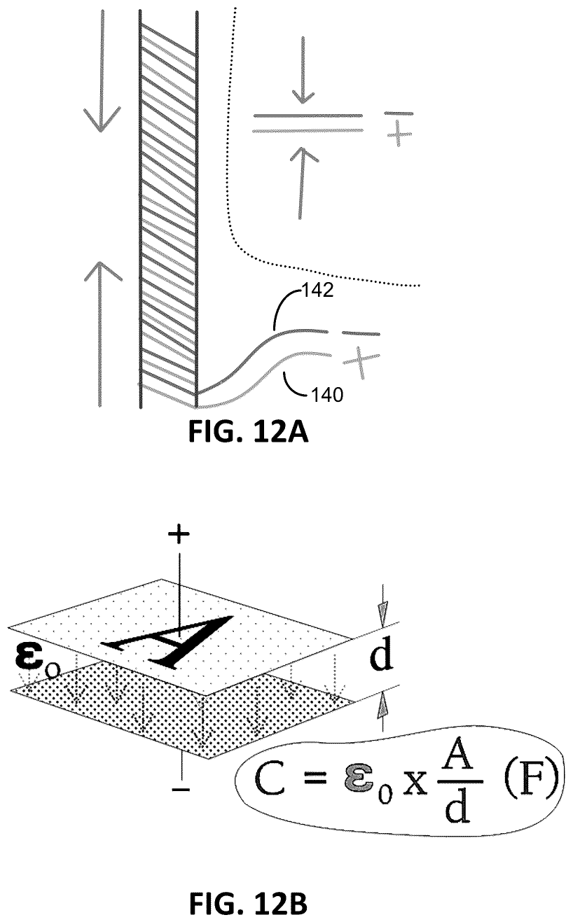

Any of a variety of sensors may be provided on any of the catheters, hubs, carriages, or table, depending upon the desired data. For example, in some implementations of the invention, it may be desirable to measure axial tension or compression force applied to the catheter such as along a force sensing zone. The distal end of the catheter would be built with a similar construction as illustrated in FIG. 11, with a helical coil distal section. But instead of using a single helical coil of nitinol wire, a first conductor 140 and second conductor 142 are wrapped into intertwined helical coils and electrically isolated from each other such as by the plastic/resin of the tubular body. See FIG. 12A. Each coil is in electrical communication with the proximal hub by a unique electrical conductor such as a conductive trace or proximal extension of the wire.

This construction of double, electrically isolated helical coils creates a capacitor. This is roughly equivalent to two plates of nitinol with a plastic layer between them, illustrated in FIG. 12B. The capacitance is inversely proportional to the distance between wires. The only variable that would be changing would be d, the distance between the plates. If an axial compressive force is applied to the catheter, the wires 140 and 142 will move closer together, thus increasing the capacitance. If an axial tensile force is applied, the wires will get further apart, decreasing the capacitance. This capacitance can be measured at the proximal end of the catheter, giving a measurement of the force at the helical capacitor. Although referred to as a capacitor, this sensor is measuring the electrical interaction between the two coils of wire. There may be a measurable change in inductance or other resulting change due to applied axial forces.

At least a first helical capacitor may have at least one or five or ten or more complete revolutions of each wire. A capacitor may be located within the distal most 5 or 10 or 20 cm of the catheter body to sense forces experienced at the distal end. At least a second capacitor may be provided within the proximal most 5 or 10 or 20 cm of the catheter body, to sense forces experienced at the proximal end of the catheter.

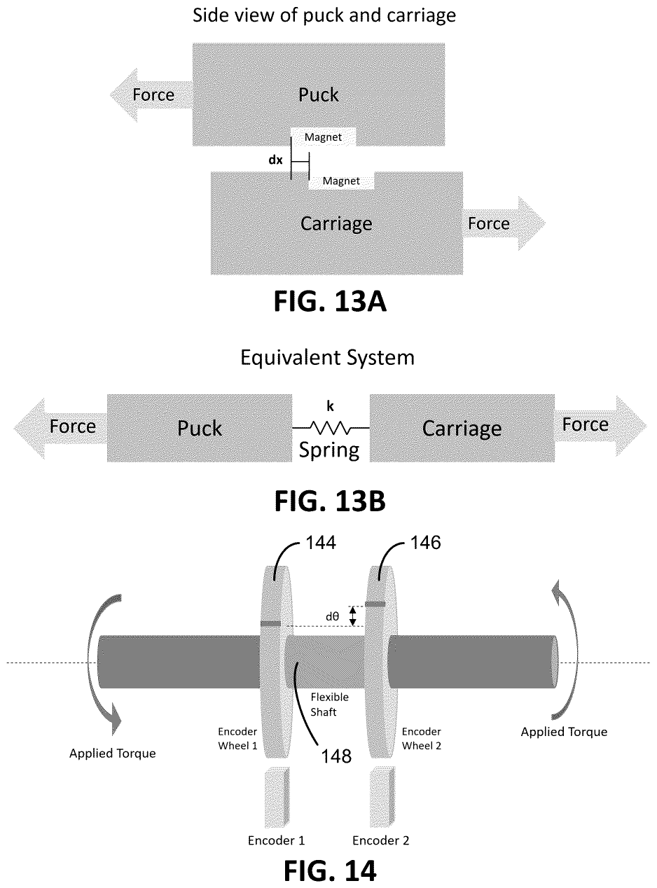

It may also be desirable to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub will move a small amount relative to the carriage. See FIG. 13A. In robotics, this is called a series elastic actuator. This property can be used to measure the force applied from the carriage to the hub. To measure the force, the relative distance between the hub and the carriage (dx shown in FIG. 13A) is determined and characterize some effective spring constant k between the two components. See FIG. 13B.

The relative distance could be measured in multiple different ways.

One method for measuring the relative distance between the puck and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magnetoresistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors would preferably be configured in a manner that avoids accumulation of blood or other fluid in the interface between the hubs carriages.

The magnetic coupling between the hub and the carriage has a break away threshold which may be about 300 grams or 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque. Such as at the distal tip and/or proximal end of the device.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the pucks through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board may run the entire working length of the table (e.g., at least about 5′ or 6′) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. The processing may be done in real time to provide position/orientation data at up to 30 hz (the max speed of the fluoro), although this technique would only provide data while the fluoro is turned on.

Proximal torque applied to the catheter or guidewire shaft may be determined using a dual encoder torque sensor. Referring to FIG. 14, a first encoder 144 and a second encoder 146 may be spaced axially apart along the shaft 148, for measuring the difference in angle over a length of flexible catheter/tube. The difference in angle is interpolated as a torque, since the catheter/tube has a known torsional stiffness. As torque is applied to the shaft, the slightly flexible portion of the shaft will twist. The difference between the angles measured by the encoders (dθ) tells us the torque. T=k*dθ, where k is the torsional stiffness.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Preferably a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path.

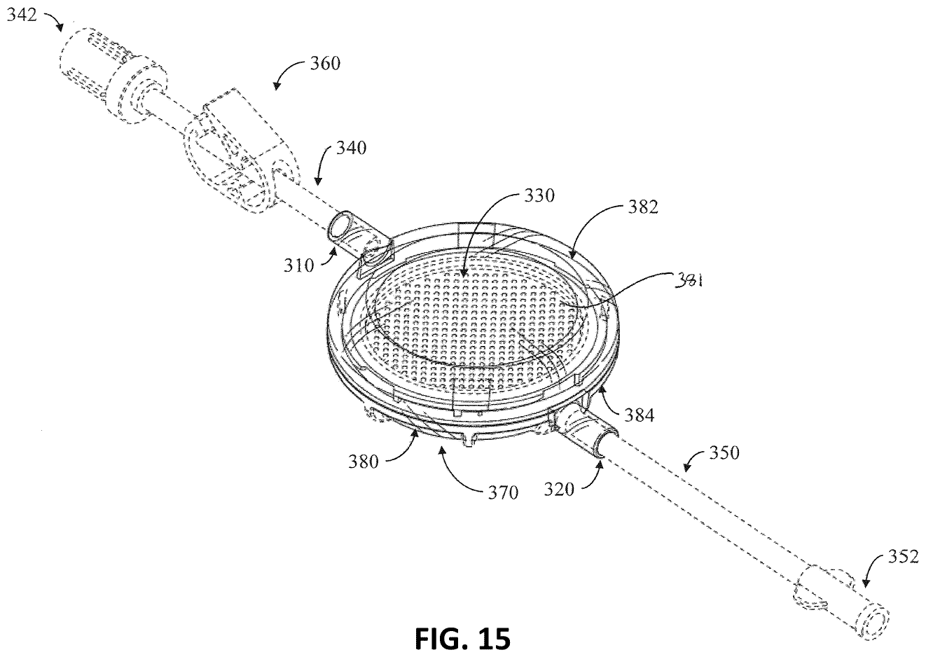

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump. Referring to FIG. 15, one example of a clot retrieval device 370 can include a body 380 enclosing a chamber 381 which communicates with a first port 310 and a second port 320. In some examples, the body 380 can include a flush port (not illustrated) that is configured to allow the injection of saline or other fluid into the chamber 381 to improve clot visualization once it is trapped in the filter 330.

In some embodiments, the body 380 includes a housing having a top portion 382 and a bottom portion 384. The body 380 may include a filter 330 positioned in the chamber 381 between the top portion 382, and the bottom portion 384. In some examples, the first port 310 is configured to connect to a first end of a first tube 340 that is fluidly connected to a proximal end of an aspiration catheter. In an embodiment that is configured to be connected downstream from the hub, the first tube 340 includes a connector 342 positioned at a second end of the first tube 340 that is configured to engage or mate with a corresponding connector on or in communication with the hub. The first port 310 directly communicates with the chamber on the upstream (e.g., top side) of the filter, and the second port 320 directly communicates with the chamber on the downstream (e.g., bottom side) of the filter to facilitate direct visualization of material caught on the upstream side of the filter. In an implementation configured for remote operation, any of a variety of sensors may be provided to detect clot passing through the aspiration line and/or trapped in the filter, such as an optical sensor, ultrasound sensor or others known in the art.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector on the pump. In some examples, the system 300 can include a clamp 360. The clamp 360 can be positioned over the first tube 340 to allow the user to engage the clamp and provide flow control over the clot retrieval device 370.

The body 380 can have a top surface spaced apart from a bottom surface by a tubular side wall. In the illustrated implementation, the top and bottom surfaces are substantially circular, and spaced apart by a cylindrical side wall having a diameter that is at least about three times, or five times or more than the axial length (transverse to the top and bottom surfaces) of the side wall, to produce a generally disc shaped housing. Preferably at least a portion of the top wall is optically transparent to improve clot visualization once it is trapped in the clot retrieval device 370. Additional details may be found in U.S. Patent Application No. 63/256,743, the entirety of each of which is hereby incorporated by reference herein.

The foregoing represents certain specific implementations of a drive table and associated catheters. a wide variety of different drive table constructions can be made, for supporting and axially advancing and retracting two or three or four or more drive magnet assemblies to robotically drive interventional devices, as will be appreciated by those of skill in the art in view of the disclosure herein.

### EXAMPLE EMBODIMENTS

A supra-aortic vessel access robotic control system comprising one or more of the following:
a guidewire hub configured to adjust each of an axial position and a rotational position of a guidewire;
a guide catheter hub configured to adjust a guide catheter in an axial direction; and
a second catheter hub configured to adjust each of an axial position and a rotational position of a second catheter, and also to laterally deflect a distal deflection zone of the second catheter.

A control system as described in any embodiment herein, wherein the second catheter is an aspiration catheter.

A control system as described in any embodiment herein, wherein the second catheter is an embolic deployment catheter.

A control system as described in any embodiment herein, wherein the second catheter is configured to deploy embolic coils.

A control system as described in any embodiment herein, wherein the second catheter is a stent deployment catheter.

A control system as described in any embodiment herein, wherein the second catheter is configured to deploy a stentriever.

A control system as described in any embodiment herein, wherein the second catheter is a flow diverter deployment catheter.

A control system as described in any embodiment herein, wherein the second catheter is a diagnostic angiographic catheter.

A control system as described in any embodiment herein, further comprising a driven magnet on the guidewire hub configured to cooperate with a drive magnet such that the driven magnet moves in response to movement of the drive magnet.

A control system as described in any embodiment herein, wherein the drive magnet is axially movably carried by a support table.

A control system as described in any embodiment herein, wherein the drive magnet moves outside of the sterile field separated from the driven magnet by a barrier, and the driven magnet is within the sterile field.

A control system as described in any embodiment herein, wherein the barrier comprises a polymer membrane.

A control system as described in any embodiment herein, further comprising a control console located remotely from the support table.

A control system as described in any embodiment herein, wherein the position of the driven magnet is movable in response to manipulation of a guidewire drive control on the console.

A control system as described in any embodiment herein, further comprising a processor for controlling the position of the driven magnet, and the processor is in wired communication with the control console.

A control system as described in any embodiment herein, further comprising a processor for controlling the position of the driven magnet, and the processor is in wireless communication with the control console.

A control system as described in any embodiment herein, wherein the driven magnet will remain engaged with the drive magnet until an applied force reaches a disruption force threshold above which the driven magnet will become decoupled from the drive magnet.

A control system as described in any embodiment herein, wherein the disruption force threshold is at least about 300 grams.

A control system as described in any embodiment herein, further comprising a sensor configured to measure the applied force between the driven magnet and the drive magnet.

A control system as described in any embodiment herein, further comprising a processor configured to compare an applied force to the disruption force threshold.

A control system as described in any embodiment herein, wherein the processor is configured to adjust a rate of movement of the drive magnet when the applied force reaches a preset value below the disruption force threshold.

A control system as described in any embodiment herein, wherein the sensor comprises a strain gauge.

A control system as described in any embodiment herein, wherein the processor is configured to halt movement of the drive magnet when the applied force reaches a preset value below the disruption force threshold.

A robotically driven interventional device comprising one or more of the following:
an elongate, flexible body, having a proximal end and a distal end;
a hub on the proximal end
at least one rotatable roller on a first surface of the hub; and
at least one magnet on the first surface of the hub.

A robotically driven interventional device as described in any embodiment herein, wherein the roller extends further away from the first surface than the magnet.

A robotically driven interventional device as described in any embodiment herein, further comprising at least a second roller.

A robotically driven interventional device as described in any embodiment herein, further comprising a rotational drive within the hub, for rotating the interventional device with respect to the hub.

A robotically driven interventional device as described in any embodiment herein, further comprising a retraction mechanism in the hub, for proximally retracting a pull element extending through the interventional device.

A robotically driven interventional device as described in any embodiment herein, wherein the pull element comprises a pull wire.

A robotically driven interventional device as described in any embodiment herein, wherein the pull element comprises a pull tube.

A robotically driven interventional device as described in any embodiment herein, wherein a shape of a portion of the tubular body changes in response to proximal retraction of the pull element.

A robotically driven interventional device as described in any embodiment herein, wherein a stiffness characteristic of a portion of the tubular body changes in response to proximal retraction of the pull element.

A robotically driven interventional device as described in any embodiment herein, further comprising a sensor on the elongate flexible body.

A robotically driven interventional device as described in any embodiment herein, wherein the sensor comprises an axial force sensor.

A robotically driven interventional device as described in any embodiment herein, wherein a distal portion of the flexible body includes at least a first electrical conductor spaced axially apart from and insulated from a second electrical conductor.

A robotically driven interventional device as described in any embodiment herein, wherein first electrical conductor and second electrical conductor are adjacent helical windings of conductive wire.

A robotically driven interventional device as described in any embodiment herein, wherein the sensor comprises an oxygen sensor.

A robotically driven interventional device as described in any embodiment herein, wherein the sensor comprises a catheter shape sensor.

A robotically driven interventional device as described in any embodiment herein, wherein the sensor comprises a catheter position sensor.

A robotically driven interventional device as described in any embodiment herein, wherein the flexible body comprises a guide catheter.

A robotically driven interventional device as described in any embodiment herein, wherein the flexible body comprises a guidewire.

A robotically driven interventional device as described in any embodiment herein, wherein the flexible body comprises an access catheter.

A robotically driven interventional device as described in any embodiment herein, wherein the flexible body comprises an aspiration catheter.

A robotically driven interventional device as described in any embodiment herein, comprising a fiber bragg grating sensor.

A robotically driven interventional device as described in any embodiment herein, further comprising a clot filter in fluid communication with the hub.

A robotically driven interventional device as described in any embodiment herein, wherein the clot filter is carried by the hub.

A robotically driven interventional device as described in any embodiment herein, wherein the clot filter has a transparent side wall to permit visual inspection of captured clot.

A robotically driven interventional device as described in any embodiment herein, further comprising a bubble detector in fluid communication with a flow path through the hub.

A robotically driven interventional device as described in any embodiment herein, wherein the bubble detector is carried by the hub.

A robotically driven interventional device as described in any embodiment herein, further comprising a valve in the flow path, and a processor configured to adjust the valve in response to detection of bubbles in the flow path.

A robotically driven interventional device as described in any embodiment herein, wherein bubbles are diverted out of the flow path in response to adjustment of the valve.

A sterile packaging assembly for transporting interventional devices to a robotic surgery site comprising one or more of the following:
a sterile barrier having a hub support portion and configured to enclose a sterile volume; and
at least a first interventional device within the sterile volume, the first interventional device including a hub and an elongate flexible body, the hub including at least one magnet and at least one roller configured to roll on the hub support portion.

A sterile packaging assembly as described in any embodiment herein, wherein the hub support portion is configured to reside on a support table adjacent a patient, with an upper surface of the hub support portion within a sterile field and a lower surface of the hub support portion outside of the sterile field.

A sterile packaging assembly as described in any embodiment herein, wherein the hub support portion is substantially horizontal when residing on the support table.

A sterile packaging assembly as described in any embodiment herein, wherein the hub support portion is inclined relative to a horizontal plane when residing on the support table.

A sterile packaging assembly as described in any embodiment herein, wherein the hub further comprises at least one fluid injection port.

A sterile packaging assembly as described in any embodiment herein, wherein the hub further comprises a wireless RF transceiver.

A sterile packaging assembly as described in any embodiment herein, further comprising a visual indicator on the hub, for indicating the presence of a clot.

A sterile packaging assembly as described in any embodiment herein, wherein the visual indicator comprises a clot collection chamber having a transparent window.

A sterile packaging assembly as described in any embodiment herein, further comprising a filter in the clot chamber.

A sterile packaging assembly as described in any embodiment herein, further comprising a sensor for detecting the presence of a clot.

A sterile packaging assembly as described in any embodiment herein, wherein the sensor comprises a pressure sensor.

A sterile packaging assembly as described in any embodiment herein, wherein the sensor comprises an optical sensor.

A sterile packaging assembly as described in any embodiment herein, wherein the hub support portion comprises an elongate polymeric membrane having a longitudinal axis.

A sterile packaging assembly as described in any embodiment herein, wherein the sterile barrier additionally comprises at least a first storage tray adjacent the hub support portion.

A sterile packaging assembly as described in any embodiment herein, comprising a first storage tray and a second storage tray adjacent the hub support portion.

A sterile packaging assembly as described in any embodiment herein, wherein the first storage tray is on a first side of the hub support portion, and the second storage tray is on a second side of the hub support portion.

A sterile packaging assembly as described in any embodiment herein, comprising a first storage tray and a second storage tray adjacent the hub support portion.

A sterile packaging assembly as described in any embodiment herein, wherein the first interventional device is contained within the first storage tray.

A sterile packaging assembly as described in any embodiment herein, wherein the first interventional device is a guide catheter.

A sterile packaging assembly as described in any embodiment herein, wherein the first interventional device is an access catheter.

A sterile packaging assembly as described in any embodiment herein, wherein the first interventional device is a guidewire.

A sterile packaging assembly as described in any embodiment herein, wherein the first interventional device is an aspiration catheter.

A sterile packaging assembly as described in any embodiment herein, comprising a supra-aortic vessel access assembly in the first storage tray.

A sterile packaging assembly as described in any embodiment herein, wherein the access assembly comprises a guidewire, an access catheter and a guide catheter.

A sterile packaging assembly as described in any embodiment herein, further comprising a procedure assembly within the sterile volume.

A sterile packaging assembly as described in any embodiment herein, wherein the procedure assembly comprises a guidewire and an aspiration catheter.

A sterile packaging assembly as described in any embodiment herein, wherein the procedure assembly is carried in a second storage tray.

A sterile packaging assembly as described in any embodiment herein, wherein the sterile barrier is magnetically permeable.

A sterile packaging assembly as described in any embodiment herein, wherein the sterile barrier is fluid impermeable.

A sterile packaging assembly as described in any embodiment herein, wherein the sterile barrier is radiofrequency permeable.

A sterile packaging assembly as described in any embodiment herein, wherein the sterile barrier is impermeable to microorganisms.

A sterile packaging assembly as described in any embodiment herein, wherein the sterile barrier is translucent.

A sterile packaging assembly as described in any embodiment herein, wherein the sterile barrier is transparent.

A sterile packaging assembly as described in any embodiment herein, wherein the hub support portion has a convex curvature such that fluid is configured to flow away from the hub support portion.

A sterile packaging assembly as described in any embodiment herein, wherein the hub support portion has a longitudinal axis and a transverse axis and the hub support portion is convex in an upward direction in the transverse axis.

A sterile packaging assembly as described in any embodiment herein, wherein the sterile barrier is contained within an outer packaging.

A sterile packaging assembly as described in any embodiment herein, wherein the sterile barrier comprises a non-compliant polymer.

A sterile packaging assembly as described in any embodiment herein, wherein the non-compliant polymer comprises Polyethylene terephthalate (PET) or a thermoplastic polyurethane.

A sterile packaging assembly as described in any embodiment herein, wherein the sterile barrier further comprises a removable cover portion that cooperates with the hub support portion to define the sterile volume.

A sterile packaging assembly as described in any embodiment herein, wherein the hub is releasably coupled to the hub support portion via the at least one magnet.

A method of performing a neurovascular procedure comprising one or more of the following steps:
providing an access catheter having an access catheter hub;
coupling the access catheter hub to a hub adapter, movably carried by a support table;
driving the access catheter in response to movement of the hub adapter along the table until the access catheter is positioned to achieve supra-aortic vessel access;
removing the access catheter and access catheter hub from the hub adapter; and
coupling a procedure catheter hub having a procedure catheter to the hub adapter.

A method as described in any embodiment herein, further comprising advancing the procedure catheter hub to position a distal end of the procedure catheter at a neurovascular treatment site.

A method as described in any embodiment herein, wherein the driving the access catheter step comprises driving the access catheter distally through a guide catheter.

A method as described in any embodiment herein, wherein the driving the access catheter step includes the step of laterally deflecting a distal region of the access catheter to achieve supra-aortic vessel access.

A method as described in any embodiment herein, wherein the coupling step comprises magnetically coupling the access catheter hub to the hub adapter.

A method as described in any embodiment herein, wherein the access catheter hub and the hub adapter are separated by a sterile field barrier.

A method as described in any embodiment herein, further comprising coupling a guide catheter hub to a guide catheter adapter through the sterile barrier.

A method as described in any embodiment herein, further comprising coupling a guidewire hub to a guidewire adapter through the sterile barrier.

A method as described in any embodiment herein, further comprising axially moving a guidewire attached to the guidewire hub in response to axially moving the guidewire adapter.

A method as described in any embodiment herein, further comprising rotating the guidewire relative to the guidewire hub.

A method as described in any embodiment herein, wherein the procedure catheter comprises an aspiration catheter.

A method as described in any embodiment herein, further comprising the step of aspirating a clot.

A method as described in any embodiment herein, further comprising driving the access catheter in response to movement of the hub adapter along the table until the access catheter achieves supra-aortic vessel access.

A method as described in any embodiment herein, further comprising maintaining supra-aortic vessel access while removing the access catheter.

A method as described in any embodiment herein, further comprising maintaining supra-aortic vessel access while coupling a procedure catheter hub.

A method as described in any embodiment herein, wherein the coupling step comprises coupling at least a first magnet on the access catheter hub to a second magnet on the hub adapter to form a magnetic coupling.

A method as described in any embodiment herein, further comprising the step of measuring elastic force across the magnetic coupling.

A method as described in any embodiment herein, further comprising the step of determining force applied to the access catheter.

A method as described in any embodiment herein, wherein the determination of force is accomplished using an optical fiber embedded in a side wall of the catheter.

A method as described in any embodiment herein, further comprising the step of determining the location of the hub adapter relative to the table.

A method of performing a neurovascular procedure, comprising one or more of the following steps:
providing an access assembly comprising a guidewire, access catheter and guide catheter;
coupling the access assembly to a robotic drive system;
driving the access assembly to achieve supra-aortic vessel access;
decoupling the guide wire and the access catheter from the access assembly;
providing a procedure assembly comprising at least a guidewire and a procedure catheter;
coupling the procedure assembly to the robotic drive system; and
performing a neurovascular procedure using the procedure assembly.

A method as described in any embodiment herein, wherein the coupling the access assembly comprises magnetically coupling a hub on each of the guidewire, access catheter and guide catheter, to separate corresponding drive magnets independently movably carried by a drive table.

A method as described in any embodiment herein, wherein the coupling the access assembly to a robotic drive system is accomplished without direct contact between the access assembly and the robotic drive system.

A method as described in any embodiment herein, wherein the procedure assembly comprises a first procedure catheter and a second procedure catheter.

A method as described in any embodiment herein, wherein the guidewire and first procedure catheter are positioned concentrically within the second procedure catheter.

A method as described in any embodiment herein, wherein the procedure assembly is advanced as a unit through at least a portion of the length of the guide catheter.

A method as described in any embodiment herein, wherein the procedure comprises a neurovascular thrombectomy.

A method as described in any embodiment herein, comprising axially advancing or retracting the guidewire.

A method as described in any embodiment herein, comprising rotating the guidewire with respect to a guidewire hub.

A method as described in any embodiment herein, comprising axially advancing or retracting the access catheter.

A method as described in any embodiment herein, comprising rotating the access catheter with respect to an access catheter hub.

A method as described in any embodiment herein, comprising laterally deflecting a deflection zone on the access catheter.

A method as described in any embodiment herein, wherein the hub on each of the guidewire, access catheter and guide catheter are separated from the corresponding drive magnets by a sterile field barrier.

A method as described in any embodiment herein, wherein driving the access assembly comprises rolling the hub on each of the guidewire, access catheter and guide catheter along the sterile field barrier in response to movement of the drive magnets.

A method as described in any embodiment herein, further comprising maintaining supra-aortic vessel access while decoupling at least one of the guide wire and the access catheter from the access assembly.

A method as described in any embodiment herein, further comprising maintaining supra-aortic vessel access while coupling the procedure assembly.

A method as described in any embodiment herein, further comprising determining relative movement between a magnet in a hub and a corresponding magnet carried by the drive table.

A method as described in any embodiment herein, further comprising determining the location of the hub relative to the drive table.

A method as described in any embodiment herein, further comprising determining axial force applied to the access catheter.

A method as described in any embodiment herein, further comprising determining rotational torque applied to the access catheter.

What is claimed is:

1. A method of performing a neurovascular procedure, comprising the steps of:
providing an access assembly comprising a guidewire, access catheter and guide catheter;
coupling the access assembly to a robotic drive system;
driving the access assembly to achieve supra-aortic vessel access;
decoupling the guidewire and the access catheter from the access assembly;
providing a procedure assembly comprising at least a guidewire and a procedure catheter;
coupling the procedure assembly to the robotic drive system; and
performing a neurovascular procedure using the procedure assembly.
